(19) United States
(12) Patent Application Publication
(10) Pub. No.: US 2022/0090299 A1
(43) Pub. Date: Mar. 24, 2022

(54) NANOFIBER AND NANOWHISKER-BASED TRANSFECTION PLATFORMS

(71) Applicant: Nanofiber Solutions, LLC, Dublin, OH (US)

(72) Inventors: Jed JOHNSON, London, OH (US); Doug KAYUHA, Dublin, OH (US); Devan OHST, Columbus, OH (US)

(73) Assignee: Nanofiber Solutions, LLC, Dublin, OH (US)

(21) Appl. No.: 17/480,254

(22) Filed: Sep. 21, 2021

Related U.S. Application Data
(60) Provisional application No. 63/081,120, filed on Sep. 21, 2020.

(51) Int. Cl. D01D 5/00; A61L 15/44; A61L 15/22; A61L 27/18; A61L 27/38

(52) U.S. Cl. CPC D01D 5/0069; A61L 15/44; B82Y 5/00; A61L 27/18; A61L 27/3839; A61L 15/22

(57) ABSTRACT

Described herein are electrospun nanofiber structures and compositions configured to serve as TNT-based platforms for the delivery of an agent or cargo, such as genetic material. The structures can include a conductive nanofiber comprising a shell electrospun from an insulating polymer, wherein the shell comprises a plurality of nanochannels therethrough, a conductive element, and an agent contained within the shell. The conductive nanofiber can be configured to deliver the agent when exposed to an electric field. The agent can include a therapeutic agent, a prophylactic agent, or a diagnostic agent.

### CROSS-REFERENCE TO RELATED APPLICATION

This application claims priority to U.S. Provisional Patent Application No. 63/081,120, titled NANOFIBER AND NANOWHISKER-BASED TRANSFECTION PLATFORMS, filed Sep. 21, 2020, which is hereby incorporated by reference herein in its entirety.

### BACKGROUND

Tissue nanotransfection (TNT) is an electroporation-based technique for delivering genes or drugs to a tissue at the nanoscale. TNT makes use of nanochannels to deliver cargo to tissues topically. TNT-based technologies can be used in combination with cell and gene therapies to, e.g., treat diseases and/or promote wound healing, as is described below.

Cell and gene therapies are a promising and growing strategy for treating a number of disorders. Animal models have been at the forefront of stem/progenitor or induced pluripotent stem cell (iPSC) therapy development and validation. Current approaches to cell and gene therapies, however, face multiple practical and translational hurdles, especially when comorbidities are considered, including the use of scarce and/or functionally impaired cell sources and the need for cumbersome and potentially immunogenic or carcinogenic ex vivo pre-processing steps (e.g., viral infection, induced pluripotency). Novel tools are thus still needed in order to enable the development, testing, and clinical implementation of highly promising cell and gene therapies.

Described herein is a disruptive nanotechnology that could establish a safer, simpler, and better-controlled approach for developing and delivering cell therapies based on direct reprogramming. Such an approach can use a single topical/non-invasive intervention lasting a brief period of time (e.g., ˜100 ms) that is capable of delivering enough reprogramming genes into tissues, so as to achieve direct non-viral conversion (i.e., bypassing induced pluripotency) of support stroma into functional parenchyma. This nanotechnology could be applicable to any species or cell type, and its non-invasive and non-viral nature makes it an ideal candidate for use in highly complex models, where systemic perturbations (e.g., inflammation, immunogenicity) could significantly impact biological responses. This technology is effective for treating disease-challenged tissues in which inherent cellular and/or microenvironmental dysfunctionalities could hamper nuclear plasticity and therapeutic efficacy. Currently, there is no example of non-viral direct adult tissue reprogramming in vivo (healthy or diseased), such as is described herein.

Recent advances in in vivo direct reprogramming have the potential to enable “on-site,” patient-specific cell therapies, which overcome major limitations by utilizing readily available cell sources, such as fibroblasts, and bypassing the need for ex vivo pre-processing and/or iPSCs. Current methodologies for in vivo reprogramming, however, are fraught with challenges, including heavy reliance on viral transfection, capsid size constraints, and high stochasticity. As such, there is still a need for safer and better-controlled non-viral methodologies for cell reprogramming. The subject matter of the present disclosure overcomes these barriers by providing a nanochannel-based transfection system with single-cell resolution that is capable of deterministic non-viral reprogramming.

Current transfection approaches (viruses, nanoparticles, or bulk electroporation (BEP)) are based on stochastic processes that lead to inefficient and/or unsafe reprogramming outcomes. BEP, in particular, is problematic because it results in variable and widespread perturbation of the cell membrane, which negatively impacts cell viability and the transfection extent.

Cell and gene therapies have also emerged as promising strategies for wound healing, especially in the presence of detrimental comorbidities (e.g., infection, diabetes, epidermolysis bullosa). Current approaches to cell and gene therapies, however, face multiple hurdles, including safety concerns due to heavy reliance on viral vectors, tumorigenesis, and immunogenicity. The nanofiber and nanowhisker-based transfection platforms described herein can be used, in at least one implementation, as part of a TNT-based wound healing system.

In addition to in vivo applications, TNT-based techniques can also be used for in vitro applications. For example, in vitro direct reprogramming of cells through the introduction of genetic material and other agents could be used to develop therapeutic cells (e.g., either to be implanted back into the subject from which the cells were harvested or other individuals) or to control the development of cultured meat products.

### SUMMARY

In one embodiment, the present disclosure is directed to a conductive nanofiber comprising: a shell electrospun from an insulating polymer, wherein the shell comprises a plurality of nanochannels therethrough; a conductive element; and an agent contained within the shell, wherein the agent comprises at least one of a therapeutic agent, a prophylactic agent, or a diagnostic agent; wherein the conductive nanofiber is configured to deliver the agent when exposed to an electric field.

In some embodiments of the conductive nanofiber, the conductive element comprises a conductive polymer fiber.

In some embodiments of the conductive nanofiber, the conductive polymer fiber comprises polyaniline.

In some embodiments of the conductive nanofiber, the agent comprises at least one of genetic material or genome editing machinery.

In some embodiments of the conductive nanofiber, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof

In some embodiments of the conductive nanofiber, the conductive nanofiber is in the form of a tubular structure.

In some embodiments of the conductive nanofiber, the conductive nanofiber is in the form of a planar structure.

In some embodiments of the conductive nanofiber, the planar structure is selected from the group consisting of a graft, a wound dressing, a muscle wrapping, and a hernia mesh.

In some embodiments of the conductive nanofiber, the insulating polymer comprises polylactide-co-glycolide acid.

In one embodiment, the present disclosure is directed to a conductive nanofiber comprising: a polymer electrospun with a plurality of conductive nanoparticles to cause the plurality of conductive nanoparticles to be blended with the polymer; and an agent loaded onto the electrospun polymer, wherein the agent comprises at least one of a therapeutic agent, a prophylactic agent, or a diagnostic agent; wherein the conductive nanofiber is configured to deliver the agent when exposed to an electric field.

In some embodiments of the conductive nanofiber, the conductive nanoparticles comprises Tantalum nanoparticles.

In some embodiments of the conductive nanofiber, the agent comprises at least one of genetic material or genome editing machinery.

In some embodiments of the conductive nanofiber, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof

In some embodiments of the conductive nanofiber, the conductive nanofiber is in the form of a tubular structure.

In some embodiments of the conductive nanofiber, the conductive nanofiber is in the form of a planar structure.

In some embodiments of the conductive nanofiber, the planar structure is selected from the group consisting of a graft, a wound dressing, a muscle wrapping, and a hernia mesh.

In one embodiment, the present disclosure is directed to a nanofiber composition comprising: a carrier medium; a plurality of electrospun nanofiber fragments or clusters; a conductive element loaded onto the plurality of nanofiber fragments or clusters; and an agent comprising at least one of a therapeutic agent, a prophylactic agent, or a diagnostic agent.

In some embodiments of the nanofiber composition, the conductive element comprises a plurality of conductive nanoparticles.

In some embodiments of the nanofiber composition, the nanofiber composition is in the form of a gel, a solution, a powder, or an aerosol.

In some embodiments of the nanofiber composition, the agent comprises genetic material or genome editing machinery.

In some embodiments of the nanofiber composition, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In one embodiment, the present disclosure is directed to a method of treating a subject, the method comprising: applying an electrospun nanofiber structure to the subject, wherein the electrospun nanofiber structure comprises a plurality of conductive nanofibers, each of the plurality of conductive nanofibers comprising: a shell electrospun from an insulating polymer, wherein the shell comprises a plurality of nanochannels therethrough, a conductive element, and an agent contained within the shell, wherein the agent comprises at least one of a therapeutic agent, a prophylactic agent, or a diagnostic agent; and applying an electric field to the electrospun nanofiber structure or the subject to cause the electrospun nanofiber structure to deliver the agent via electroporation of cell membranes of the subject.

In some embodiments of the method of treating the subject, the conductive element comprises a conductive polymer fiber.

In some embodiments of the method of treating the subject, the conductive polymer fiber comprises polyaniline.

In some embodiments of the method of treating the subject, the agent comprises at least one of genetic material or genome editing machinery.

In some embodiments of the method of treating the subject, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In some embodiments of the method of treating the subject, the conductive nanofiber is in the form of a tubular structure.

In some embodiments of the method of treating the subject, the conductive nanofiber is in the form of a planar structure.

In some embodiments of the method of treating the subject, the planar structure is selected from the group consisting of a graft, a wound dressing, a muscle wrapping, and a hernia mesh.

In some embodiments of the method of treating the subject, the insulating polymer comprises polylactide-co-glycolide acid.

In one embodiment, the present disclosure is directed to a method of treating a subject, the method comprising: applying an electrospun nanofiber structure to the subject, wherein the electrospun nanofiber structure comprises a plurality of conductive nanofibers, each of the plurality of conductive nanofibers comprising: a polymer electrospun with a plurality of conductive nanoparticles to cause the plurality of conductive nanoparticles to be blended with the polymer, and an agent loaded onto the electrospun polymer, wherein the agent comprises at least one of a therapeutic agent, a prophylactic agent, or a diagnostic agent; and applying an electric field to the electrospun nanofiber structure or the subject to cause the electrospun nanofiber structure to deliver the agent via electroporation of cell membranes of the subject.

In some embodiments of the method of treating the subject, the conductive nanoparticles comprises Tantalum nanoparticles.

In some embodiments of the method of treating the subject, the agent comprises at least one of genetic material or genome editing machinery.

In some embodiments of the method of treating the subject, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In some embodiments of the method of treating the subject, the electrospun nanofiber structure is in the form of a tubular structure.

In some embodiments of the method of treating the subject, the electrospun nanofiber structure is in the form of a planar structure.

In some embodiments of the method of treating the subject, the planar structure is selected from the group consisting of a graft, a wound dressing, a muscle wrapping, and a hernia mesh.

In one embodiment, the present disclosure is directed to a method of treating a subject, the method comprising: applying an electrospun nanofiber composition to the subject, wherein the electrospun nanofiber structure comprises: a carrier medium, a plurality of nanofiber fragments or clusters, a conductive element loaded onto the plurality of nanofiber fragments or clusters, and an agent comprising at least one of a therapeutic agent, a prophylactic agent, or a diagnostic agent; and applying an electric field to the electrospun nanofiber composition or the subject to cause the electrospun nanofiber composition to deliver the agent via electroporation of cell membranes of the subject.

In some embodiments of the method of treating the subject, the conductive element comprises a plurality of conductive nanoparticles.

In some embodiments of the method of treating the subject, the nanofiber composition is in the form of a gel, a solution, a powder, or an aerosol.

In some embodiments of the method of treating the subject, the agent comprises genetic material or genome editing machinery.

In some embodiments of the method of treating the subject, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In one embodiment, the present disclosure is directed to a method of fabricating an electrospun nanofiber structure, the method comprising: dissolving an insulating polymer in a first solvent, wherein the solvent is immiscible in water; electrospinning the insulating polymer from an outer needle at a high relative humidity; electrospinning a conductive polymer from an inner needle, wherein the inner needle is arranged coaxially with respect to the inner needle; wherein the electrospun insulating polymer forms a shell of the electrospun nanofiber structure and the electrospun conductive polymer forms a core of the electrospun nanofiber structure, wherein the core is contained within the shell; dissolving the solvent to form a plurality of nanochannels in the shell; and applying an agent to the electrospun nanofiber structure, wherein the agent comprises at least one of a therapeutic agent, a prophylactic agent, or a diagnostic agent.

In some embodiments of the method of fabricating the electrospun nanofiber structure, the conductive polymer fiber comprises polyaniline.

In some embodiments of the method of fabricating the electrospun nanofiber structure, the agent comprises at least one of genetic material or genome editing machinery.

In some embodiments of the method of fabricating the electrospun nanofiber structure, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In some embodiments of the method of fabricating the electrospun nanofiber structure, the conductive nanofiber is in the form of a tubular structure.

In some embodiments of the method of fabricating the electrospun nanofiber structure, the conductive nanofiber is in the form of a planar structure.

In some embodiments of the method of fabricating the electrospun nanofiber structure, the planar structure is selected from the group consisting of a graft, a wound dressing, a muscle wrapping, and a hernia mesh.

In some embodiments of the method of fabricating the electrospun nanofiber structure, the insulating polymer comprises polylactide-co-glycolide acid.

In one embodiment, the present disclosure is directed to a method of fabricating an electrospun nanofiber structure, the method comprising: dissolving an insulating polymer in a solvent, wherein the solvent is immiscible in water; electrospinning the insulating polymer at a high relative humidity; dissolving the solvent to form the electrospun nanofiber structure comprising a plurality of nanochannels; and applying a conductive element and an agent to the electrospun nanofiber structure, wherein the agent comprises at least one of a therapeutic agent, a prophylactic agent, or a diagnostic agent.

In some embodiments of the method of fabricating the electrospun nanofiber structure, the conductive nanoparticles comprises Tantalum nanoparticles.

In some embodiments of the method of fabricating the electrospun nanofiber structure, the agent comprises at least one of genetic material or genome editing machinery.

In some embodiments of the method of fabricating the electrospun nanofiber structure, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

In some embodiments of the method of fabricating the electrospun nanofiber structure, the electrospun nanofiber structure is in the form of a tubular structure.

In some embodiments of the method of fabricating the electrospun nanofiber structure, the electrospun nanofiber structure is in the form of a planar structure.

In some embodiments of the method of fabricating the electrospun nanofiber structure, the planar structure is selected from the group consisting of a graft, a wound dressing, a muscle wrapping, and a hernia mesh.

In one embodiment, the present disclosure is directed to a method of fabricating an electrospun nanofiber composition, the method comprising: electrospinning a polymer to form an electrospun nanofiber structure; pulverizing the electrospun nanofiber structure to form a plurality of nanofiber fragments or clusters; loading a conductive element onto the plurality of nanofiber fragments or clusters; and adding the plurality of nanofiber fragments or clusters loaded with the conductive element and an agent comprising at least one of a therapeutic agent, a prophylactic agent, or a diagnostic agent to a carrier medium to form the electrospun nanofiber composition.

In some embodiments of the method of fabricating the electrospun nanofiber structure, the conductive element comprises a plurality of conductive nanoparticles.

In some embodiments of the method of fabricating the electrospun nanofiber structure, the nanofiber composition is in the form of a gel, a solution, a powder, or an aerosol.

In some embodiments of the method of fabricating the electrospun nanofiber structure, the agent comprises genetic material or genome editing machinery.

In some embodiments of the method of fabricating the electrospun nanofiber structure, the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

### BRIEF DESCRIPTION OF THE FIGURES

The accompanying drawings, which are incorporated in and form a part of the specification, illustrate the embodiments of the invention and together with the written description serve to explain the principles, characteristics, and features of the invention.

FIG. 1 illustrates a diagram of an electrospun nanofiber structure in accordance with at least one aspect of the present disclosure.

FIG. 2 illustrates a scanning electron microscope (SEM) image of 10 wt % polylactide-co-glycolide acid (PLG) 82:18 dissolved in dichloromethane (DCM) and then electrospun in a nanofiber sheet at 2350× magnification showing the nanoporous surface in accordance with at least one aspect of the present disclosure.

FIG. 3 illustrates a SEM image of electrospun 100 wt % tantalum (Ta) of 10 wt % PLG 82:18 dissolved in DCM and then electrospun into a nanofiber sheet at 2450× magnification in accordance with at least one aspect of the present disclosure.

FIG. 4 illustrates a SEM image of core-shell electrospun 1000 wt % Ta of 6 wt % PLG 82:18 dissolved in hexaflouroisoproponol (HFIP) (for the core) and 10 wt % PLG 82:18+DCM (for the shell) and then electrospun into a nanofiber sheet at 1750× magnification in accordance with at least one aspect of the present disclosure.

FIG. 5 illustrates a photograph of an experimental TNT setup using the structures and/or compositions described herein in accordance with at least one aspect of the present disclosure.

### DETAILED DESCRIPTION

This disclosure is not limited to the particular systems, devices and methods described, as these may vary. The terminology used in the description is for the purpose of describing the particular versions or embodiments only, and is not intended to limit the scope of the disclosure.

The following terms shall have, for the purposes of this application, the respective meanings set forth below. Unless otherwise defined, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the singular forms “a,” “an,” and “the” include plural references, unless the context clearly dictates otherwise. Thus, for example, reference to a “fiber” is a reference to one or more fibers and equivalents thereof known to those skilled in the art, and so forth.

As used herein, the term “about” means plus or minus 10% of the numerical value of the number with which it is being used. Therefore, about 50 mm means in the range of 45 mm to 55 mm.

As used herein, the term “consists of” or “consisting of” means that the device or method includes only the elements, steps, or ingredients specifically recited in the particular claimed embodiment or claim.

In embodiments or claims where the term “comprising” is used as the transition phrase, such embodiments can also be envisioned with replacement of the term “comprising” with the terms “consisting of” or “consisting essentially of.”

The terms “animal,” “patient,” and “subject” as used herein include, but are not limited to, humans and non-human vertebrates such as wild, domestic, and farm animals. In some embodiments, the terms “animal,” “patient,” and “subject” may refer to humans.

As used herein, the term “biocompatible” refers to non-harmful compatibility with living tissue. Biocompatibility is a broad term that describes a number of materials, including bioinert materials, bioactive materials, bioabsorbable materials, biostable materials, biotolerant materials, or any combination thereof.

As used herein, the term “nanowhisker” refers to electrospun nanofiber fragments, clusters, and/or combinations thereof.

As used herein, the term “fragment” refers to a portion of a particular fiber. In some embodiments, a fragment may comprise at least one polymer, having an average length of about 1 μm to about 1000 μm, and an average diameter of about 0.1 μm to about 10 μm. In some embodiments, a composition may contain a plurality of fragments. In some embodiments, a composition may contain a plurality of fragments and, optionally, a carrier medium. In some embodiments, a composition may contain a plurality of fragments, a carrier medium, and, optionally, a plurality of cells. Some non-limiting examples of average fragment lengths may include an average length of about 1 μm, an average length of about 5 μm, an average length of about 10 μm, an average length of about 20 μm, an average length of about 30 μm, an average length of about 40 μm, an average length of about 50 μm, an average length of about 75 μm, an average length of about 90 μm, an average length of about 95 μm, an average length of about 100 μm, an average length of about 105 μm, an average length of about 110 μm, an average length of about 150 μm, an average length of about 200 μm, an average length of about 300 μm, an average length of about 400 μm, an average length of about 500 μm, an average length of about 600 μm, an average length of about 700 μm, an average length of about 800 μm, an average length of about 900 μm, an average length of about 1000 μm, or ranges between any two of these values (including endpoints). Some non-limiting examples of average fragment diameters may include an average diameter of about 0.1 μm, an average diameter of about 0.5 μm, an average diameter of about an average diameter of about 2 μm, an average diameter of about 3 μm, an average diameter of about 4 μm, an average diameter of about 5 μm, an average diameter of about 6 μm, an average diameter of about 7 μm, an average diameter of about 8 μm, an average diameter of about 9 μm, an average diameter of about 10 μm, or ranges between any two of these values (including endpoints). When combined with a carrier medium, the resulting mixture may include from about 1 fragment per mm³ to about 100,000 fragments per mm³. Some non-limiting examples of mixture densities may include about 2 fragments per mm³, about 100 fragments per mm³, about 1,000 fragments per mm³, about 2,000 fragments per mm³, about 5,000 fragments per mm³, about 10,000 fragments per mm³, about 20,000 fragments per mm³, about 30,000 fragments per mm³, about 40,000 fragments per mm³, about 50,000 fragments per mm³, about 60,000 fragments per mm³, about 70,000 fragments per mm³, about 80,000 fragments per mm³, about 90,000 fragments per mm³, about 100,000 fragments per mm³, or ranges between any two of these values (including endpoints).

As used herein, the term “cluster” refers to an aggregate of fiber fragments, or a linear or curved three-dimensional group of fiber fragments. In some embodiments, a cluster may comprise at least one polymer. Clusters may have a range of shapes. Non-limiting examples of cluster shapes may include spherical, globular, ellipsoidal, and flattened cylinder shapes. Clusters may have, independently, an average length of about 1 μm to about 1000 μm, an average width of about 1 μm to about 1000 μm, and an average height of about 1 μm to about 1000 μm. It may be appreciated that any cluster dimension, such as length, width, or height, is independent of any other cluster dimension. Some non-limiting examples of average cluster dimensions include an average dimension (length, width, height, or other measurement) of about an average dimension of about 5 μm, an average dimension of about 10 μm, an average dimension of about 20 μm, an average dimension of about 30 μm, an average dimension of about 40 μm, an average dimension of about 50 μm, an average dimension of about 75 μm, an average dimension of about 90 μm, an average dimension of about 95 μm, an average dimension of about 100 μm, an average dimension of about 105 μm, an average dimension of about 110 μm, an average dimension of about 150 μm, an average dimension of about 200 μm, an average dimension of about 300 μm, an average dimension of about 400 μm, an average dimension of about 500 μm, an average dimension of about 600 μm, an average dimension of about 700 μm, an average dimension of about 800 μm, an average dimension of about 900 μm, an average dimension of about 1000 μm, or ranges between any two of these values (including endpoints), or independent combinations of any of these ranges of dimensions. Clusters may include an average number of about 2 to about 1000 fiber fragments. Some non-limiting examples of average numbers of fiber fragments per cluster include an average of about 2 fiber fragments per cluster, an average of about 5 fiber fragments per cluster, an average of about 10 fiber fragments per cluster, an average of about 20 fiber fragments per cluster, an average of about 30 fiber fragments per cluster, an average of about 40 fiber fragments per cluster, an average of about 50 fiber fragments per cluster, an average of about 60 fiber fragments per cluster, an average of about 70 fiber fragments per cluster, an average of about 80 fiber fragments per cluster, an average of about 90 fiber fragments per cluster, an average of about 100 fiber fragments per cluster, an average of about 110 fiber fragments per cluster, an average of about 200 fiber fragments per cluster, an average of about 300 fiber fragments per cluster, an average of about 400 fiber fragments per cluster, an average of about 500 fiber fragments per cluster, an average of about 600 fiber fragments per cluster, an average of about 700 fiber fragments per cluster, an average of about 800 fiber fragments per cluster, an average of about 900 fiber fragments per cluster, an average of about 1000 fiber fragments per cluster, or ranges between any two of these values (including endpoints). In some embodiments, a composition may contain a plurality of clusters. In some embodiments, a composition may contain a plurality of fragments and a plurality of clusters. In some embodiments, a composition may contain a plurality of fragments, a plurality of clusters, and, optionally, a carrier medium. In some embodiments, a composition may contain a plurality of fragments, a plurality of clusters, a carrier medium, and, optionally, a plurality of cells.

As used herein, the term “nanochannels” means a channel or pore that is on the nanometer size scale.

As used herein, the term “therapeutic” means an agent utilized to treat, combat, ameliorate, prevent, or improve an unwanted condition or disease of a patient. In part, embodiments of the present disclosure are directed to the treatment of wounds, injuries of tendons, ligaments, or other musculoskeletal structures, organs, and the like.

A “therapeutically effective amount” or “effective amount” of a composition is a predetermined amount calculated to achieve the desired effect, i.e., to improve, localize, increase, inhibit, block, or reverse the adhesion, activation, migration, penetration, or proliferation of cells. The activity contemplated by the present methods includes medical, therapeutic, cosmetic, aesthetic, and/or prophylactic treatment, as appropriate. The specific dose of a compound administered according to this disclosure to obtain therapeutic, cosmetic, aesthetic, and/or prophylactic effects will, of course, be determined by the particular circumstances surrounding the case, including, for example, the compound administered, the route of administration, and the condition being treated. The compounds are effective over a wide dosage range. It will be understood that the effective amount administered will be determined by the physician, veterinarian, or other medical professional in the light of the relevant circumstances including the condition to be treated, the choice of compound to be administered, and the chosen route of administration, and therefore the dosage ranges described herein are not intended to limit the scope of the disclosure in any way.

The terms “treat,” “treated,” or “treating” as used herein refer to both therapeutic treatment and prophylactic or preventative measures, wherein the object is to prevent or slow down (lessen) or entirely reverse (eradicate) an undesired physiological condition, disorder or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; remission (whether partial or total), whether detectable or undetectable, or enhancement or improvement of the condition, disorder, or disease; and eradication of the condition, disorder, or disease. Treatment includes eliciting a clinically significant response without excessive levels of side effects. Treatment also includes prolonging survival as compared to expected survival if not receiving treatment.

### Electrospinning Fibers

Electrospinning is a method which may be used to process a polymer solution into a fiber. In embodiments where the diameter of the resulting fiber is on the nanometer scale, the fiber may be referred to as a nanofiber. Fibers may be formed into a variety of shapes by using a range of receiving surfaces, such as mandrels or collectors. In some embodiments, a flat shape, such as a sheet or sheet-like fiber mold, a fiber scaffold and/or tube, or a tubular lattice, may be formed by using a substantially round or cylindrical mandrel. In certain embodiments, the electrospun fibers may be cut and/or unrolled from the mandrel as a fiber mold to form the sheet. The resulting fiber molds or shapes may be used in many applications, including the repair or replacement of biological structures. In some embodiments, the resulting fiber scaffold may be implanted into a biological organism or a portion thereof.

Electrospinning methods may involve spinning a fiber from a polymer solution by applying a high DC voltage potential between a polymer injection system and a mandrel. In some embodiments, one or more charges may be applied to one or more components of an electrospinning system. In some embodiments, a charge may be applied to the mandrel, the polymer injection system, or combinations or portions thereof. Without wishing to be bound by theory, as the polymer solution is ejected from the polymer injection system, it is thought to be destabilized due to its exposure to a charge. The destabilized solution may then be attracted to a charged mandrel. As the destabilized solution moves from the polymer injection system to the mandrel, its solvents may evaporate, and the polymer may stretch, leaving a long, thin fiber that is deposited onto the mandrel. The polymer solution may form a Taylor cone as it is ejected from the polymer injection system and exposed to a charge.

In certain embodiments, a first polymer solution comprising a first polymer and a second polymer solution comprising a second polymer may each be used in a separate polymer injection system at substantially the same time to produce one or more electrospun fibers comprising the first polymer interspersed with one or more electrospun fibers comprising the second polymer. Such a process may be referred to as “co-spinning” or “co-electrospinning,” and a scaffold produced by such a process may be described as a co-spun or co-electrospun scaffold.

### Polymer Injection System

A polymer injection system may include any system configured to eject some amount of a polymer solution into an atmosphere to permit the flow of the polymer solution from the injection system to the mandrel. In some embodiments, the polymer injection system may deliver a continuous or linear stream with a controlled volumetric flow rate of a polymer solution to be formed into a fiber. In some embodiments, the polymer injection system may deliver a variable stream of a polymer solution to be formed into a fiber. In some embodiments, the polymer injection system may be configured to deliver intermittent streams of a polymer solution to be formed into multiple fibers. In some embodiments, the polymer injection system may include a syringe under manual or automated control. In some embodiments, the polymer injection system may include multiple syringes and multiple needles or needle-like components under individual or combined manual or automated control. In some embodiments, a multi-syringe polymer injection system may include multiple syringes and multiple needles or needle-like components with each syringe containing the same polymer solution. In some embodiments, a multi-syringe polymer injection system may include multiple syringes and multiple needles or needle-like components with each syringe containing a different polymer solution. In some embodiments, a charge may be applied to the polymer injection system or to a portion thereof. In some embodiments, a charge may be applied to a needle or needle-like component of the polymer injection system.

In some embodiments, the polymer solution may be ejected from the polymer injection system at a flow rate of less than or equal to about 5 mL/h per needle. In other embodiments, the polymer solution may be ejected from the polymer injection system at a flow rate per needle in a range from about 0.01 mL/h to about 50 mL/h. The flow rate at which the polymer solution is ejected from the polymer injection system per needle may be, in some non-limiting examples, about 0.01 mL/h, about 0.05 mL/h, about 0.1 mL/h, about 0.5 mL/h, about 1 mL/h, about 2 mL/h, about 3 mL/h, about 4 mL/h, about 5 mL/h, about 6 mL/h, about 7 mL/h, about 8 mL/h, about 9 mL/h, about 10 mL/h, about 11 mL/h, about 12 mL/h, about 13 mL/h, about 14 mL/h, about 15 mL/h, about 16 mL/h, about 17 mL/h, about 18 mL/h, about 19 mL/h, about 20 mL/h, about 21 mL/h, about 22 mL/h, about 23 mL/h, about 24 mL/h, about 25 mL/h, about 26 mL/h, about 27 mL/h, about 28 mL/h, about 29 mL/h, about 30 mL/h, about 31 mL/h, about 32 mL/h, about 33 mL/h, about 34 mL/h, about 35 mL/h, about 36 mL/h, about 37 mL/h, about 38 mL/h, about 39 mL/h, about 40 mL/h, about 41 mL/h, about 42 mL/h, about 43 mL/h, about 44 mL/h, about 45 mL/h, about 46 mL/h, about 47 mL/h, about 48 mL/h, about 49 mL/h, about 50 mL/h, or any range between any two of these values, including endpoints.

As the polymer solution travels from the polymer injection system toward the mandrel, the diameter of the resulting fibers may be in the range of about 0.1 μm to about 10 μm. Some non-limiting examples of electrospun fiber diameters may include about 0.1 μm, about 0.2 μm, about 0.25 μm, about 0.5 μm, about 1 μm, about 2 μm, about 5 μm, about 10 μm, about 20 μm, or ranges between any two of these values, including endpoints. In some embodiments, the electrospun fiber diameter may be from about 0.25 μm to about 20 μm.

### Polymer Solution

In some embodiments, the polymer injection system may be filled with a polymer solution. In some embodiments, the polymer solution may comprise one or more polymers. In some embodiments, the polymer solution may be a fluid formed into a polymer liquid by the application of heat. A polymer solution may include, for example, non-resorbable polymers, resorbable polymers, natural polymers, or a combination thereof.

In some embodiments, the polymers may include, for example, polyethylene terephthalate, polyurethane, polyethylene, polyethylene oxide, polyester, polymethylmethacrylate, polyacrylonitrile, silicone, polycarbonate, polyether ketone ketone, polyether ether ketone, polyether imide, polyamide, polystyrene, polyether sulfone, polysulfone, polyvinyl acetate, polytetrafluoroethylene, polyvinylidene fluoride, polycaprolactone, polylactic acid, polyglycolic acid, polylactide-co-glycolide, polylactide-co-caprolactone, polyglycerol sebacate, polydioxanone, polyhydroxybutyrate, poly-4-hydroxybutyrate), trimethylene carbonate, polydiols, polyesters, collagen, gelatin, fibrin, fibronectin, albumin, hyaluronic acid, elastin, chitosan, alginate, silk, copolymers thereof, and combinations thereof.

It may be understood that polymer solutions may also include a combination of one or more of non-resorbable, resorbable polymers, and naturally occurring polymers in any combination or compositional ratio. In an alternative embodiment, the polymer solutions may include a combination of two or more non-resorbable polymers, two or more resorbable polymers or two or more naturally occurring polymers. In some non-limiting examples, the polymer solution may comprise a weight percent ratio of, for example, from about 5% to about 90%. Non-limiting examples of such weight percent ratios may include about 5%, about 10%, about 15%, about 20%, about 25%, about 30%, about 33%, about 35%, about 40%, about 45%, about 50%, about 55%, about 60%, about 66%, about 70%, about 75%, about 80%, about 85%, about 90%, or ranges between any two of these values, including endpoints.

In some embodiments, the polymer solution may comprise one or more solvents. In some embodiments, the solvent may comprise, for example, acetone, dimethylformamide, dimethylsulfoxide, N-methylpyrrolidone, N,N-dimethylformamide, Nacetonitrile, hexanes, ether, dioxane, ethyl acetate, pyridine, toluene, xylene, tetrahydrofuran, trifluoroacetic acid, hexafluoroisopropanol, acetic acid, dimethylacetamide, chloroform, dichloromethane, water, alcohols, ionic compounds, or combinations thereof. The concentration range of polymer or polymers in solvent or solvents may be, without limitation, from about 1 wt % to about 50 wt %. Some non-limiting examples of polymer concentration in solution may include about 1 wt %, 3 wt %, 5 wt %, about 10 wt %, about 15 wt %, about 20 wt %, about 25 wt %, about 30 wt %, about 35 wt %, about 40 wt %, about 45 wt %, about 50 wt %, or ranges between any two of these values, including endpoints.

In some embodiments, the polymer solution and/or the resultant electrospun polymer fiber(s) may also include additional materials. Non-limiting examples of such additional materials may include radiation opaque materials, contrast agents, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, other materials to aid in non-invasive imaging, or any combination thereof. In some embodiments, the radiation opaque materials may include, for example, barium, tantalum, tungsten, iodine, gadolinium, gold, platinum, bismuth, or bismuth (III) oxide. In some embodiments, the electrically conductive materials may include, for example, gold, silver, iron, or polyaniline.

In some embodiments, the additional materials may be present in the polymer solution in an amount from about 1 wt % to about 1500 wt % of the polymer mass. In some non-limiting examples, the additional materials may be present in the polymer solution in an amount of about 1 wt %, about 5 wt %, about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %, about 100 wt %, about 125 wt %, about 150 wt %, about 175 wt %, about 200 wt %, about 225 wt %, about 250 wt %, about 275 wt %, about 300 wt %, about 325 wt %, about 350 wt %, about 375 wt %, about 400 wt %, about 425 wt %, about 450 wt %, about 475 wt %, about 500 wt %, about 525 wt %, about 550 wt %, about 575 wt %, about 600 wt %, about 625 wt %, about 650 wt %, about 675 wt %, about 700 wt %, about 725 wt %, about 750 wt %, about 775 wt %, about 800 wt %, about 825 wt %, about 850 wt %, about 875 wt %, about 900 wt %, about 925 wt %, about 950 wt %, about 975 wt %, about 1000 wt %, about 1025 wt %, about 1050 wt %, about 1075 wt %, about 1100 wt %, about 1125 wt %, about 1150 wt %, about 1175 wt %, about 1200 wt %, about 1225 wt %, about 1250 wt %, about 1275 wt %, about 1300 wt %, about 1325 wt %, about 1350 wt %, about 1375 wt %, about 1400 wt %, about 1425 wt %, about 1450 wt %, about 1475 wt %, about 1500 wt %, or any range between any of these two values, including endpoints. In one embodiment, the polymer solution may include tantalum present in an amount of about 10 wt % to about 1,500 wt %.

The type of polymer in the polymer solution may determine the characteristics of the electrospun fiber. Some fibers may be composed of polymers that are bio-stable and not absorbable or biodegradable when implanted. Such fibers may remain generally chemically unchanged for the length of time in which they remain implanted. Alternatively, fibers may be composed of polymers that may be absorbed or bio-degraded over time. Such fibers may act as an initial template or scaffold during a healing process. These templates or scaffolds may degrade in vivo once the tissues have a degree of healing by natural structures and cells. It may be further understood that a polymer solution and its resulting electrospun fiber(s) may be composed of more than one type of polymer, and that each polymer therein may have a specific characteristic, such as bio-stability, biodegradability, or bioabsorbability.

### Applying Charges to Electrospinning Components

In an electrospinning system, one or more charges may be applied to one or more components or portions of components, such as, for example, a mandrel or a polymer injection system or a portion thereof. In some embodiments, a positive charge may be applied to the polymer injection system or a portion thereof. In some embodiments, a negative charge may be applied to the polymer injection system or a portion thereof. In some embodiments, the polymer injection system, or a portion thereof, may be grounded. In some embodiments, a positive charge may be applied to a mandrel or a portion thereof. In some embodiments, a negative charge may be applied to the mandrel or a portion thereof. In some embodiments, the mandrel, or a portion thereof, may be grounded. In some embodiments, one or more components, or portions thereof, may receive the same charge. In some embodiments, one or more components, or portions thereof, may receive one or more different charges.

The charge applied to any component of the electrospinning system, or any portion thereof, may be from about −15 kV to about 30 kV, including endpoints. In some non-limiting examples, the charge applied to any component of the electrospinning system, or any portion thereof, may be about −15 kV, about −10 kV, about −5 kV, about −4 kV, about −3 kV, about −1 kV, about −0.01 kV, about 0.01 kV, about 1 kV, about 5 kV, about 10 kV, about 11 kV, about 11.1 kV, about 12 kV, about 15 kV, about 20 kV, about 25 kV, about 30 kV, or any range between any two of these values, including endpoints. In some embodiments, any component of the electrospinning system, or any portion thereof, may be grounded.

### Mandrel Movement During Electrospinning

During electrospinning, in some embodiments, the mandrel may move with respect to the polymer injection system. In some embodiments, the polymer injection system may move with respect to the mandrel. The movement of one electrospinning component with respect to another electrospinning component may be, for example, substantially rotational, substantially translational, or any combination thereof. In some embodiments, one or more components of the electrospinning system may move under manual control. In some embodiments, one or more components of the electrospinning system may move under automated control. In some embodiments, the mandrel may be in contact with or mounted upon a support structure that may be moved using one or more motors or motion control systems. The pattern of the electrospun fiber deposited on the mandrel may depend upon the one or more motions of the mandrel with respect to the polymer injection system. In some embodiments, the mandrel surface may be configured to rotate about its long axis. In one non-limiting example, a mandrel having a rotation rate about its long axis that is faster than a translation rate along a linear axis, may result in a nearly helical deposition of an electrospun fiber, forming windings about the mandrel. In another example, a mandrel having a translation rate along a linear axis that is faster than a rotation rate about a rotational axis, may result in a roughly linear deposition of an electrospun fiber along a liner extent of the mandrel.

### Electrospun Nanofiber Fragments and Clusters

Nanofiber structures may also be processed into small fragments and aggregates of fragments, or clusters. In one embodiment, nanofiber fragments and/or clusters may be prepared by freezing an electrospun nanofiber structure, for example in liquid nitrogen. Freezing the electrospun nanofiber structure may result in increased brittleness, resulting in structures that may be readily pulverized into small fragments. Pulverization techniques may include, without limitation, grinding, chopping, pulverizing, micronizing, milling, shearing, or any combination thereof. Fragments may have an average length of about 10 μm to about 1000 μm. In one non-limiting example, fragments may have an average length of about 100 μm. Such pulverized electrospun compositions may also be compressed into fiber suspensions. In one non-limiting example, the compressed fiber suspension may be pelletized, or otherwise formed into a compressed or pellet-like structure.

Such fragments or clusters may be initially prepared by the processes described herein, followed by one or a range of pulverizing procedures as described above. Such fragments or clusters may be resorbable, non-resorbable, or a combination thereof. Fragments may have an average length of about 1 μm to about 1000 μm. In one non-limiting example, fragments may have an average length of about 100 μm. Clusters may have a range of shapes. Non-limiting examples of cluster shapes include spherical, globular, ellipsoidal, and flattened cylinder shapes. Clusters may have, independently, an average length of about 1 μm to about 1000 μm, an average width of about 1 μm to about 1000 μm, and an average height of about 1 μm to about 1000 μm, and may include an average number of about 2 to about 1000 fiber fragments. In one non-limiting example, clusters may include an average number of about 100 fiber fragments. In some embodiments, the electrospun nanofiber fragments and/or clusters may be used to retain or localize cells or other components incorporated therewith, to promote cell infusion, attachment, adhesion, penetration, or proliferation, to stimulate cell or tissue growth, healing, or, in some cases, shrinkage, or any combination of uses thereof.

Such electrospun nanofiber fragments and/or clusters may be fabricated from a polymer solution as described above. The polymer solution may include additional materials. In a non-limiting example, electrospun nanofiber fragments and/or clusters may be manufactured or impregnated with additional materials, which the fragments and/or clusters may later elute. Non-limiting examples of such additional materials may include radiation opaque materials, electrically conductive materials, fluorescent materials, luminescent materials, antibiotics, growth factors, vitamins, cytokines, steroids, anti-inflammatory drugs, small molecules, sugars, salts, peptides, proteins, cell factors, DNA, RNA, any materials to aid in non-invasive imaging, or any combination thereof. Non-limiting examples of radiation opaque materials may include barium, tantalum, tungsten, iodine, or gadolinium. Non-limiting examples of electrically conductive materials may include gold, silver, iron, or polyaniline.

Such electrospun nanofiber fragments and/or clusters may be added to a carrier medium to produce a suspension for delivery to a body part or system. The suspension may have a volume of about 0.1 mL to about 50 mL. The suspension may also comprise electrospun nanofiber fragments and/or clusters in a weight percent to carrier medium of about 0.001 wt % to about 50 wt %. In some non-limiting examples, the carrier medium may be phosphate buffered saline, cell culture media, platelet-rich plasma, plasma, lactated Ringer's solution, a gel, a powder, an aerosol, or any combination thereof. In some non-limiting examples, the suspension may be injected into a joint. Non-limiting examples of joints in which the suspension may be injected may include the knee, the shoulder, and the hip. In one non-limiting example, the suspension may be injected using a syringe with a 20-gauge needle. In some non-limiting examples, the suspension may be injected into a tendon or ligament. In some non-limiting examples, the suspension may be injected intravenously, intramuscularly, subcutaneously, or intraperitoneally. In some non-limiting examples, the suspension may be delivered topically. In one non-limiting example, the suspension may be applied topically to a wound. In some non-limiting examples, the suspension may be inserted during surgery. In some non-limiting examples, the suspension may be delivered by ingestion, inhalation, or suppository. In some non-limiting examples, the suspension may be printed into a construct or scaffold. In one non-limiting example, the suspension may be printed, such as via a three-dimensional printer, for eventual application in the body or a system.

The above-described suspensions of electrospun nanofiber fragments and/or clusters may include additional components along with the carrier medium. Non-limiting examples of additional bioactive components may include antibiotics, tissue growth factors, platelet-rich plasma, amnion, small molecules, or any combination thereof. Biologically active cells may also be included in the suspensions. Biologically active cells may include differentiated cells, stem cells, or any combination thereof. Such biologically active cells may be added to the suspensions to provide cells for improved repair of injured or stunted tissues. Stem cells may include multipotent stem cells, pluripotent stem cells, and totipotent stem cells. Such stem cells may be autologous (from the same patient), syngeneic (from an identical twin, if available), allogeneic (from a non-patient donor), or any combination thereof. In some non-limiting embodiments, the stem cells may include adult stem cells such as bone marrow-derived stem cells, cord blood stem cells, or mesenchymal cells. Other types of stem cells may include embryonic stem cells or induced pluripotent stem cells. It may be appreciated that a suspension of electrospun nanofiber fragments and/or clusters in a carrier medium may incorporate adult stem cells, embryonic stem, induced pluripotent stem cells, differentiated cells, or any combination thereof.

Electrospun nanofiber fragments and/or clusters may be combined with other carrier materials and are not limited to purely aqueous suspensions. In some other non-limiting embodiments, micronized nanofiber textile fragments may be combined with gels, pastes, powders, aerosols, and/or other carriers. In one non-limiting example, the nanofiber fragments and/or clusters may be combined with a carrier capable of forming a gel, solid, powder, or aerosol when implanted into a recipient (human or non-human animal). Gelation or solidification of the carrier may occur on exposure of the suspension to the biological environment due, for example, to a change in temperature or pH. Alternative carriers may include components capable of responding to externally applied stimuli such as magnetic fields, electric fields, or sonic fields. In one non-limiting example, a carrier may respond to an applied magnetic field to cause the textile fragments to orient in a specific direction. Electrospun nanofiber fragments and/or clusters without a carrier may also be implanted in a recipient. In one non-limiting application, electrospun nanofiber fragments and/or clusters may be implanted directly into a solid tumor. The implanted fragments and/or clusters may concentrate externally applied heat, sonic, or radiation energy to the tumor. In one non-limiting example, electrospun nanofiber fragments and/or clusters may be implanted for the purpose of localized or systemic delivery of drugs, biological materials, contrast agents, or other materials as disclosed above.

In one non-limiting example, electrospun nanofiber fragments and/or clusters may be sold in a kit. In a non-limiting example, the kit may further comprise a carrier medium. In a non-limiting example, the kit may further comprise instructions for the use of the electrospun nanofiber fragments, clusters, and/or carrier medium. In a non-limiting example, the carrier medium may be any of the above-disclosed carrier media, in any form, including, for example, a gel, a dry form such as a powder, an aerosol, a liquid, or any other form, including those which may be reconstituted for use.

### Electrospun Polymer Fibers for Cultured Meat Production

Scaffolds of various sizes and thicknesses may help solve the engineering problems that cultured meat products currently face. In general, using a cellular engineering process that involves cells and such a scaffold may allow for the migration of the cells throughout the entirety of the scaffold. However, many existing scaffolds fail to provide the correct representation of the extracellular matrix.

Electrospun polymer fibers may provide solutions to these challenges. Electrospun polymer fibers may be used to create scaffolds of various sizes and thicknesses. In contrast to scaffolds made from other materials, electrospun polymer fibers may be formed into a variety of shapes, including discs, tubes, sheets, and the like, making them easy to fit into existing cell culture devices. The use of electrospun polymer fiber scaffolds may allow the creation of a higher volume of cultured meat using existing equipment. Moreover, electrospun fiber scaffolds could be used to develop products with specific structures (including meats such as steaks or sashimi, for example), targeting a specific volume and cellular environment for the final product. Electrospun polymer fibers can be used, for example, to create a scaffold having highly aligned fibers. Such aligned fibers may provide the necessary topographical and electrical cues to cells in culture, thereby providing appropriate stimulation for the development of engineered musculoskeletal tissue.

Furthermore, and without wishing to be bound by theory, it is thought that some of the taste in traditional slaughtered meat is the result of lactate or lactic acid. Lactic acid is produced in two instances: in times of high stress and during anaerobic respiration. Research has suggested that post-mortem, muscle cells continue to operate for a short period of time from anaerobic respiration. The lactic acid produced during that period is thought to drop the pH of the meat to around 5.5, although a wider range of pH values may be found in different meats. Electrospun polymer fibers can be engineered to specifically deteriorate or dissolve over a period of time into chemical byproducts naturally found in the body, including lactic acid, glycolic acid, and caproic acid. The period of time can range depending on the planned end product, and can be anywhere from about 1 day to about 6 weeks. The dissolution of electrospun polymer fibers into these chemical byproducts may create a more acidic environment that would lead to an improved cultured meat product. A small drop in the pH of the cell environment may also encourage healthy, organized tissue growth. Accordingly, a decrease in pH during culturing could lead to improved tissue growth (and thereby improved texture), as well as improved taste of the cultured meat product.

Furthermore, electrospun polymer fibers may be made from various different polymers, as described above, and these different polymers may be used to promote different cell differentiation and/or proliferation properties for different components of cultured meat, including myocytes, adipocytes, and chondrocytes in muscle, fat, and connective tissue, respectively. These different tissue types differentiate stem cells in their own unique ways based on different environmental and/or chemical signals. Electrospun polymer fibers could be used to create a scaffold having different sections with different properties, each section designed to generate and support a desired tissue type. Electrospun polymer fibers can be manufactured with different moduli, diameters, surface textures, surface chemical interactions, or spatially controlled drug delivery systems. In short, electrospun polymer fibers could be used to create cultured meat products that look, feel, and taste like traditional slaughtered meats.

In some embodiments, the cultured meat products described herein may comprise a scaffold and a population of cells. The population of cells may include, in some non-limiting examples, mesenchymal stem cells, myocytes, adipocytes, chondrocytes, osteoblasts, or any combination thereof. Publications that demonstrate the culture of myocytes, adipocytes chondrocytes, and osteoblasts on electrospun polymer fibers include: (1) Khan et al., Evaluation of Changes in Morphology and Function of Human Induced Pluripotent Stem Cell Derived Cardiomyocytes (HiPSC-CMs) Cultured on an Aligned-Nanofiber Cardiac Patch. PLOS One. 2015; 10(5):e0126338. doi:10.1371/journal/pone.0126338, which is incorporated herein by reference in its entirety; and (2) Pandey et al., Aligned Nanofiber Material Supports Cell Growth and Increases Osteogenesis in Canine Adipose-Derived Mesenchymal Stem Cells In Vitro. J Biomed Mater Res Part A 2018, 106A:1780-1788, which is incorporated herein by reference in its entirety.

The scaffold may comprise an electrospun polymer fiber as described herein. In some embodiments, the electrospun polymer fiber may comprise a polymer selected from nylon, nylon 6,6, polycaprolactone, polyethylene oxide terephthalate, polybutylene terephthalate, polyethylene oxide terephthalate-co-polybutylene terephthalate, polyethylene terephthalate, polyurethane, polyethylene, polyethylene oxide, polyvinylpyrrolidone, polymethylmethacrylate, polyacrylonitrile, silicone, polycarbonate, polylactide, polyglycolide, polyether ketone ketone, polyether ether ketone, polyether imide, polyamide, polystyrene, polyether sulfone, polysulfone, polyvinyl acetate, polytetrafluoroethylene, polyvinylidene fluoride, polylactic acid, polyglycolic acid, polylactide-co-glycolide, poly(lactide-co-caprolactone), polyglycerol sebacate, polydioxanone, polyhydroxybutyrate, poly-4-hydroxybutyrate, trimethylene carbonate, polydiols, polyesters, collagen, gelatin, fibrin, fibronectin, albumin, hyaluronic acid, elastin, chitosan, alginate, silk, zein, a soy protein, a plant protein, a carbohydrate, copolymers thereof, and combinations thereof. In some embodiments, the resulting electrospun polymer fiber may include a combination of one or more of a plant protein, a carbohydrate, and a synthetic polymer.

In certain embodiments, the electrospun polymer fiber may comprise multiple electrospun polymer fibers aligned substantially parallel to one another, as described herein. In other embodiments, the electrospun fiber may comprise multiple electrospun polymer fibers having different orientations relative to one another, including randomly oriented, substantially parallel, and combinations thereof, as described herein. In embodiments having multiple electrospun polymer fibers, the multiple electrospun polymer fibers may have multiple orientations and/or multiple fiber diameters, as described herein, and may comprise one or more polymers, as described herein. In certain embodiments, a scaffold may comprise multiple co-spun electrospun polymer fibers, as described herein.

In some embodiments, the scaffold may further comprise one or more electrospun polymer fiber fragments. The electrospun polymer fiber fragments may be, for example, dispersed throughout the scaffold or dispersed throughout a particular portion of the scaffold. Without wishing to be bound by theory, the electrospun polymer fiber fragments may aid or support the culturing and expansion of cells within the scaffold. In some embodiments, the electrospun polymer fiber fragments may have a length from about 100 μm to about 10 mm. In certain embodiments, the electrospun polymer fiber fragments may have a maximum length of about 1 mm.

In certain embodiments, the scaffold may comprise one or more electrospun polymer fiber types, and the one or more electrospun polymer fiber types may be co-spun. In an embodiment, each electrospun fiber type may be suitable to support the differentiation of one or more cells into a different biological tissue. For example, a scaffold may comprise a first electrospun polymer fiber type suitable to support the differentiation of cells into muscle, a second electrospun polymer fiber type suitable to support the differentiation of cells into bone, a third electrospun polymer fiber type suitable to support the differentiation of cells into cartilage, a fourth electrospun polymer fiber type suitable to support the differentiation of cells into a connective tissue, a fifth electrospun polymer fiber type suitable to support the differentiation of cells into a blood vessel, or any combination of these electrospun polymer fiber types.

A scaffold may include, in one non-limiting example, a first plurality of electrospun polymer fibers comprising a polymer and having a diameter and/or orientation to support the proliferation of a first type of cells; a second plurality of electrospun polymer fibers comprising a polymer and having a diameter and/or orientation to support the proliferation of a second type of cells; a third plurality of electrospun polymer fibers comprising a polymer and having a diameter and/or orientation to support the proliferation of a third type of cells; a fourth plurality of electrospun polymer fibers comprising a polymer and having a diameter and/or orientation to support the proliferation of a fourth type of cells; and so on. In some embodiments, the first, second, third, and fourth types of cells in such embodiments may include any mammalian cells, such as muscle cells, vascular cells, fat cells, connective tissue cells, neural cells, or combinations thereof.

In some embodiments, the electrospun polymer fiber may comprise a polymer configured to degrade to produce a byproduct. In certain embodiments, the byproduct may include, for example, lactic acid, glycolic acid, caproic acid, and combinations thereof. In some embodiments, the electrospun polymer fiber may be configured to degrade upon exposure to a substance; in one non-limiting example, the substance may comprise saliva.

In certain embodiments, the electrospun polymer fiber may comprise an additional material, as described herein, and may be configured to release at least a portion of the additional material upon the application of a mechanical force. In one embodiment, the mechanical force may be produced by actions such as chewing, cutting, breaking, or combinations thereof. In some embodiments, the cultured meat product may include an intact electrospun polymer fiber, while in other embodiments, the electrospun polymer fiber of the scaffold may be completely or nearly completely resorbed in the final cultured meat product. In an embodiment, the intact electrospun polymer fiber may be configured to mimic the texture and/or other properties of traditional slaughtered meat.

In certain embodiments, the cultured meat product may have a thickness from about 100 μm to about 500 mm. The thickness may be, for example, about 100 μm, about 200 μm, about 300 μm, about 400 μm, about 500 μm, about 600 μm, about 700 μm, about 800 μm, about 900 μm, about 1 mm, about 5 mm, about 10 mm, about 25 mm, about 50 mm, about 75 mm, about 100 mm, about 125 mm, about 150 mm, about 175 mm, about 200 mm, about 225 mm, about 250 mm, about 275 mm, about 300 mm, about 325 mm, about 350 mm, about 375 mm, about 400 mm, about 425 mm, about 450 mm, about 475 mm, about 500 mm, or any range between any two of these values, including endpoints. In some embodiments, the cultured meat product may have a thickness from about 5 mm to about 75 mm. In an embodiment, the thickness may be about 25 mm.

In some embodiments, the cultured meat products described herein may be configured to mimic or closely resemble a property of a traditional slaughtered meat. The property may include, for example, taste, texture, size, shape, topography, or any combination thereof.

In some embodiments, a method of producing a cultured meat product may comprise preparing a scaffold as described herein, placing the scaffold into a bioreactor, adding a population of cells to the bioreactor, culturing the population of cells in the bioreactor containing the scaffold for a period of time, thereby forming the cultured meat product, and removing the cultured meat product from the bioreactor. In embodiments, the cultured meat product may have the characteristics and features of the cultured meat products described herein. In some embodiments, the scaffold and population of cells may each have the characteristics and features of the scaffolds and populations of cells described herein.

In some embodiments, the step of culturing the population of cells in the bioreactor may be carried out for a period of time. The period of time could be, for example, about 1 day, about 2 days, about 3 days, about 4 days, about 5 days, about 6 days, about 1 week, about 1.5 weeks, about 2 weeks, about 2.5 weeks, about 3 weeks, about 3.5 weeks, about 4 weeks, about 4.5 weeks, about 5 weeks, about 5.5 weeks, about 6 weeks, or any range between any two of these values, including endpoints. In one embodiment, the period of time may be about 3 weeks.

### Nanofiber Scaffold Structures for Tissue Nanotransfection

The instant disclosure is directed to nanofiber structures, such as scaffolds, and/or compositions for delivering one or more agents, such as genetic material, using TNT-based techniques. TNT is an emergent technology capable of highly efficient non-viral delivery of gene and/or cell therapies to tissues. Incorporating TNT capabilities into nanofiber scaffolds enables simultaneous delivery of powerful gene/cell therapies. Described herein are electrospun nanofiber structures and compositions including conducting elements and nanochanneled surfaces that are suitable for TNT-based delivery of genetic material and other agents. In some embodiments, these structures and/or compositions can be embodied as electrically conductive wound dressings, which enable electroceutical wound therapies. In some embodiments, TNT-capable fibers can be pulverized into nanowhiskers, which can be injected for deep gene/cell therapy and/or applied to topical or surgical wounds (e.g., via spraying or sprinkling) for the treatment thereof. In still other embodiments, the structures and/or compositions can be used to control the development of cultured meat products through the delivery of genetic material and other agents thereto.

In some embodiments, the electrospun nanofiber structures and/or compositions described herein provide a non-viral approach to topically and controllably deliver reprogramming genes to tissues through a nanochanneled platform based on TNT techniques. Such approach allows direct gene delivery in a rapid (e.g., ˜100 milliseconds) and non-invasive manner by applying a highly intense and focused electric field through nanochannels, which benignly nanoporates the juxtaposing cell membranes, and electrophoretically drives the genes into cells. Nanochannel-based poration is highly uniform and confined to a small portion (<0.1%) of the cell membrane. This approach is highly beneficial compared to BEP because BEP results in variable and widespread perturbation of the cell membrane, which negatively impacts cell viability and the transfection extent. Consequently, nanochannel-based delivery results in stronger gene expression compared to BEP.

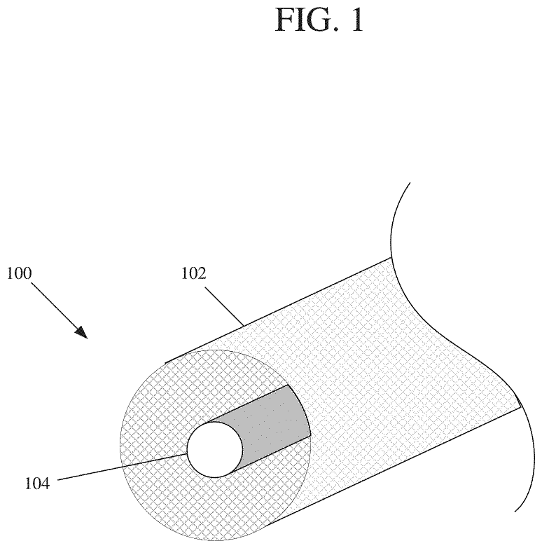

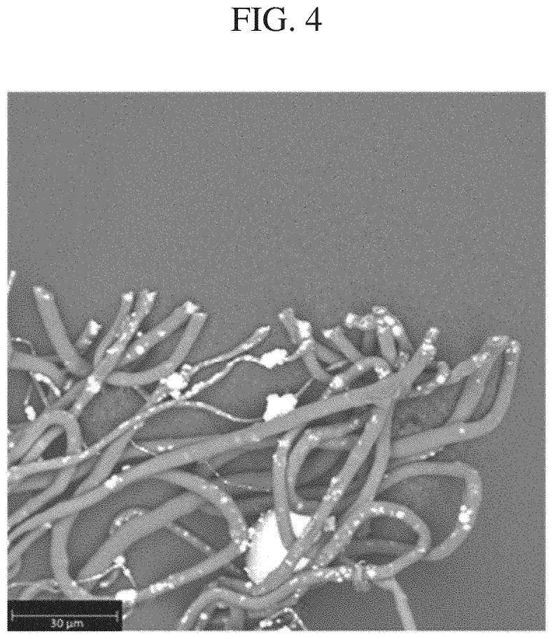

In various embodiments, an electrospun nanofiber structure can be fabricated from electrospun fibers that include a conductive element and one or more agents, such as genetic material, to use in the TNT-based delivery of the one or more agents. FIG. 1 illustrates a general diagram of one such embodiment of an electrospun nanofiber 100. These electrospun nanofibers 100 can be part or a component of a larger structure or device (e.g., a graft) with other such fibers. In this embodiment, the electrospun nanofiber 100 can include a shell 102 and a conductive element 104. In one embodiment, the shell 102 can include or be fabricated from an insulating polymer. In various embodiments, the insulating polymer can include PLG. In one embodiment, the conductive element can include a conductive polymer fiber 104 that includes or is fabricated from a conductive polymer, as is shown in FIG. 1. In various embodiments, the conductive polymer can include polyaniline (PANi). In one illustrative embodiment, the insulating polymer can include PLG and the conductive polymer can include PANi. The conductive polymer fiber 104 can be contained within or otherwise enclosed by the shell 102, for example. In one particular embodiment, the shell 102 can be embodied as an elongated structure and the conductive polymer fiber 104 can likewise be embodied as an elongated structure extending through the shell 102. In one embodiment, the conductive polymer can be core/shell-electrospun with an insulating polymer to form the conductive polymer fiber 104 and the shell 102 in conjunction with each other, such as is described below in connection with FIG. 4.

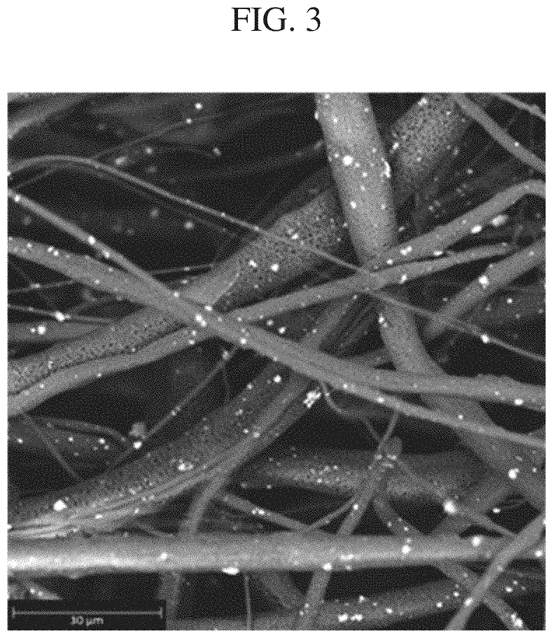

In another embodiment, rather than having the nanofibers electrospun in the core/shell configuration that is illustrated in FIG. 1, the conductive element can include conductive particles (e.g., Ta) that are loaded onto or blended into the polymer from which the nanofibers are electrospun, as shown in FIG. 3. In one embodiment, the conductive particles can be added to the polymer solution from which the shell 102 is electrospun. In another embodiment, the conductive particles can be loaded into a monolithic fiber.

In yet another embodiment, conductive electrospun nanofibers can include a combination of multiple conductive elements. For example, the electrospun nanofibers could include both a shell 102 (FIG. 1) including conductive particles and a conductive polymer fiber 104 enclosed within the shell 102.

Using the techniques described above, the various embodiments of electrospun nanofibers can be manufactured into structures having a variety of different shapes and/or configurations, such as a tubular structure or a planar structure (i.e., a sheet). Tubular electrospun nanofiber structures fabricated from these conductive electrospun nanofibers could be used as, e.g., neural grafts, neural conduits, or synthetic organs (e.g., an esophagus or trachea) or portions thereof. Embodiments where the conductive electrospun nanofibers are manufactured into sheets could be used as, e.g., grafts (e.g., rotator cuff grafts), wound dressings (for either topical wounds or surgical wounds), wrappings for muscles, or implantable devices (e.g., hernia meshes). In addition to tubular structures and sheets, the conductive electrospun nanofibers can be manufactured into structures having a variety of other shapes and configurations.

In the embodiments where the conductive electrospun nanofibers are core/shell-spun nanofibers, the shell 102 is configured to isolate the conductive polymer fiber 104 from the subject's surrounding tissue, which may lead to improved cell viability post-transfection as compared to blended fibers. In embodiments where conductive electrospun nanofibers include a polymer blended with conductive particles (e.g., such as PLG blended with Ta nanoparticles, as shown in FIG. 3), such blended fibers may result in electric field maximization at the fiber surface and thus improved electrophoretic motility of the plasmid DNA and transfection outcomes. However, although such configurations can result in stronger electric fields and improved transfection, this may come at the expense of tissue viability. Accordingly, one could elect to use the core/shell-spun nanofiber embodiments or the nanoparticle-loaded nanofiber embodiments based upon whether it is desirable to improve cell viability post-transfection or improve transfection outcomes, for example.

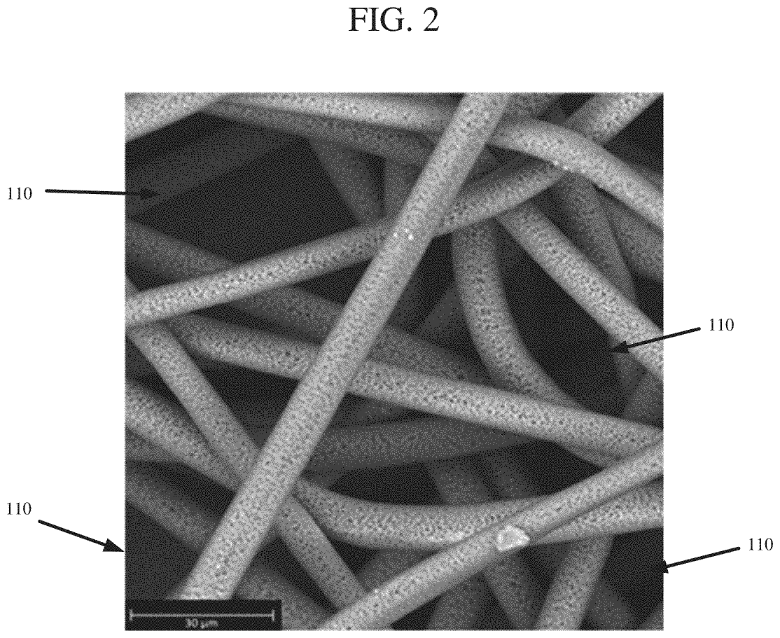

In various embodiments, the conductive electrospun nanofibers can be manufactured to include nanochannels 110 (also referred to as “nanopores”), such as are shown in FIG. 2. The nanochannels 110 are beneficial for TNT-based applications of the conductive electrospun nanofibers because the nanochannels 110 focus the electric field applied to the electrospun nanofiber structures to precise points, which minimizes the amount of stress imparted on the cell membranes of the tissue. This is in stark contrast to BEP, which puts a substantial amount of stress on the cell membranes, resulting in negative impacts to cell viability and the transfection extent. In general, electrospinning creates smooth surfaces, so certain techniques must be used in order to create the nanochannels 110 in the electrospun nanofiber structure. In one embodiment, the polymer to be electrospun is dissolved in a solvent that is immiscible with water, such as DCM. Further, the electrospinning is performed at a high (e.g., >40%) relative humidity. Electrospinning with an immiscible solvent at high relative humidity causes a phase separation at the surface of the electrospun structure, creating solvent-rich regions and solvent-poor (i.e., polymer-rich) regions. When the solvent is evaporated at the completion of the electrospinning, the solvent-rich regions leave pores (i.e., the nanochannels 110) in the resulting electrospun fiber. Various other techniques for creating nanochannels 110 are described in “Novel Electrospun Scaffolds for the Molecular Analysis of Chondrocytes Under Dynamic Compression,” Nam et al., Tissue Engineering Part A, vol. 15(3) (March 2009), 513-23, which is hereby incorporated by reference herein in its entirety. In some embodiments, the nanochannels 110 can be used in conjunction with either the core/shell-spun nanofiber embodiments or the nanoparticle-loaded nanofiber embodiments. In the core/shell-spun nanofiber embodiments, the nanochannels 110 can be located on the shell 102 in order to assist in focusing the electric field propagated by the conductive element (e.g., the conductive polymer fiber 104).

In various embodiments, the electrospun nanofibers can further include one or more therapeutic, prophylactic, or diagnostic agents. For the embodiments of the core/shell-spun nanofibers 100, the one or more agents can be positioned within the shell 102, for example. In one embodiment, the one or more agents (e.g., genetic cargo) can be coated, loaded, or otherwise applied to the conductive fibers or the structure comprising the conductive fibers after they have been electrospun using various techniques known in the art, such as using sub-critical CO₂, super-critical CO₂, adsorption, and so on. Various techniques can be used to load the agents onto or into the nanofibers. For example, in some embodiments, the agents can be added into the polymer solution before electrospinning nanofibers or can be electrospun as a component of the shell 102 (for the core/shell-spun embodiments) using a coaxial needle setup. For example, the nanofiber structures described herein could be electrospun using a needle assembly comprising a first or outer needle and a second or inner needle, wherein the inner needle is arranged coaxially with respect to the outer needle. In this example, the outer needle can include a first polymer (e.g., an insulating polymer) and the inner needle can include a second polymer (e.g., a conductive polymer). Using this needle assembly, a nanofiber 100 can be fabricated by simultaneously electrospinning the first and second polymers from the outer and inner needles, respectively, to form the shell 102 and the conductive element 104 structures, as shown in FIG. 1. In some embodiments, the agents can be adsorbed onto the surface of the nanofibers after electrospinning by soaking the fibers in a solution of the cargo. In some embodiments, the agents can be embedded into the nanofibers via subcritical/supercritical CO₂. Once loaded, the fibers can be formulated for topical or internal delivery, such as injection or infusion, in the form of a solution or suspension. The formulation can be administered via any route, such as, the blood stream or directly to the organ or tissue to be treated via implantation.

In one embodiment, the one or more agents can include genetic material, such as DNA, RNA, cDNA, extrachromosomal DNA (e.g., plasmids), messenger RNA (mRNA), ribosomal RNA (rRNA), transfer RNA (tRNA), small interfering RNA (siRNA), micro RNA (miRNA), small nuclear RNA (snRNA), small nucleolar RNA (snoRNA), piwi-interacting RNA (piRNA), non-coding RNA (ncRNA), long noncoding RNA (lncRNA), fragments or portions thereof, and the like. The genetic material can correspond to particular genes that are desired to be introduced to the subject's tissue or isolated cells using TNT-based techniques, for example. In another embodiment, the one or more agents can include genome editing machinery.

In another embodiment, the nanofiber compositions disclosed herein can include a carrier medium, a plurality of nanowhiskers, a conductive element, and one or more therapeutic, prophylactic, or diagnostic agents. The nanowhiskers can be manufactured using any of the techniques described above. Further, the conductive element can include any of the conductive elements described above. In particular, a structure can be electrospun from an insulating polymer using the techniques described above. In one embodiment, the electrospun structure can be loaded with conductive particles using the techniques described above. The electrospun structure loaded with the conductive particles can then be processed (e.g., frozen and then pulverized) using the techniques described above to generate nanofiber fragments and/or clusters loaded with the conductive particles. In another embodiment, the electrospun structure can be processed to generate nanofiber fragments and/or clusters and the resulting fragments and/or clusters can be loaded with the conductive particles using the techniques described above. The one or more agents can be combined with the nanowhiskers and conductive elements within a carrier medium, for example, to form a composition. The carrier medium can include both aqueous and non-aqueous carrier mediums, for example. Accordingly, the compositions described herein could be embodied as liquids, gels, powders, solids, or aerosols, for example. As described above, the one or more agents could include genetic material. In some embodiments, the nanofiber compositions described herein could be injected into or applied topically to a subject. After being applied to the subject, an electric field could be applied to the subject and/or the composition, and the composition could facilitate the TNT-based delivery of the agent (e.g., genetic material) to the subject.

In one embodiment, a method of fabricating an electrospun nanofiber structure can include: (i) dissolving an insulating polymer in a first solvent, wherein the solvent is immiscible in water; (ii) electrospinning the insulating polymer from an outer needle at a high relative humidity; (iii) electrospinning a conductive polymer from an inner needle, wherein the inner needle is arranged coaxially with respect to the inner needle, wherein the electrospun insulating polymer forms a shell of the electrospun nanofiber structure and the electrospun conductive polymer forms a core of the electrospun nanofiber structure, wherein the core is contained within the shell; (iv) dissolving the solvent to form a plurality of nanochannels in the shell; and (v) applying an agent to the electrospun nanofiber structure, wherein the agent comprises at least one of a therapeutic agent, a prophylactic agent, or a diagnostic agent. In one embodiment, a method of fabricating an electrospun nanofiber structure can include: (i) dissolving an insulating polymer in a solvent, wherein the solvent is immiscible in water; (ii) electrospinning the insulating polymer at a high relative humidity; (iii) dissolving the solvent to form the electrospun nanofiber structure comprising a plurality of nanochannels; and (iv) applying a conductive element and an agent to the electrospun nanofiber structure, wherein the agent comprises at least one of a therapeutic agent, a prophylactic agent, or a diagnostic agent. In one embodiment, a method of fabricating an electrospun nanofiber composition can include: (i) electrospinning a polymer to form an electrospun nanofiber structure; (ii) pulverizing the electrospun nanofiber structure to form a plurality of nanofiber fragments or clusters; (iii) loading a conductive element onto the plurality of nanofiber fragments or clusters; and (iv) adding the plurality of nanofiber fragments or clusters loaded with the conductive element and an agent comprising at least one of a therapeutic agent, a prophylactic agent, or a diagnostic agent to a carrier medium to form the electrospun nanofiber composition.

As noted above, TNT requires that an electric field be applied to the subject to induce electroporation in the subject's cell membranes to facilitate delivery of the agent. In the embodiments described herein that are applied topically to the subject, the electric field could be applied across the electrospun nanofiber structure or nanofiber composition. The conductive elements would then selectively induce electroporation in the subject's cells (e.g., as focused by the nanochannels in the nanofiber structure or the nanoparticles), thereby facilitating delivery of the agent. For example, in one particular embodiment, the electrospun nanofiber structure could be embodied as a sheet configured for use as a wound dressing. In this particular embodiment, the electrospun sheet could be applied over a subject's wound, and an electric field could be applied across the electrospun sheet resulting in delivery of the agent to the subject's wound and other surrounding tissue. In the embodiments described herein that are injected into, implanted into, or otherwise applied internally to the subject, the electric field could be applied across the subject to facilitate the delivery of the agent, as described above.

In one embodiment, a method of treating a subject can include: (i) applying any of the various embodiments electrospun nanofiber structures described above to the subject, such as an electrospun nanofiber structure that comprises a plurality of conductive nanofibers, each of the plurality of conductive nanofibers comprising: a shell electrospun from an insulating polymer, wherein the shell comprises a plurality of nanochannels therethrough, a conductive element, and an agent contained within the shell, wherein the agent comprises at least one of a therapeutic agent, a prophylactic agent, or a diagnostic agent; and (ii) applying an electric field to the electrospun nanofiber structure or the subject to cause the electrospun nanofiber structure to deliver the agent via electroporation of cell membranes of the subject. In one embodiment, a method of treating a subject can include: (i) applying any of the various embodiments electrospun nanofiber structures described above to the subject, such as an electrospun nanofiber structure that comprises a plurality of conductive nanofibers, each of the plurality of conductive nanofibers comprising: a polymer electrospun with a plurality of conductive nanoparticles to cause the plurality of conductive nanoparticles to be blended with the polymer, and an agent loaded onto the electrospun polymer, wherein the agent comprises at least one of a therapeutic agent, a prophylactic agent, or a diagnostic agent; and (ii) applying an electric field to the electrospun nanofiber structure or the subject to cause the electrospun nanofiber structure to deliver the agent via electroporation of cell membranes of the subject. In one embodiment, a method of treating a subject, the method comprising: (i) applying any of the various embodiments electrospun nanofiber compositions described above to the subject, such as electrospun nanofiber compositions that comprises: a carrier medium, a plurality of nanofiber fragments or clusters, a conductive element loaded onto the plurality of nanofiber fragments or clusters, and an agent comprising at least one of a therapeutic agent, a prophylactic agent, or a diagnostic agent; and (ii) applying an electric field to the electrospun nanofiber composition or the subject to cause the electrospun nanofiber composition to deliver the agent via electroporation of cell membranes of the subject.

In some embodiments, the electrospun nanofiber structures and compositions described herein can be used in the context of cultured meat products. In particular, the structures and compositions described herein can be used to deliver genetic material or other agents to cells being cultured to produce meat products in order to control the development of the cultured meat products. As one example application, a biopsy could be taken from an animal of choice and the animal's cells could be expanded in the laboratory. However, the harvested cells and the cells proliferating in the lab may not be the desired final cell type for the cultured meat product. Accordingly, the structures and/or compositions described herein could be used with TNT-based techniques to introduce agents (e.g., genetic material) to differentiate the harvested cells to the desired cell types in a very efficient manner.

Various concepts of the structures, compositions, and techniques described above are illustrated using specific examples, which are set forth below. These examples are meant solely to illustrate the concepts described above and are not intended to be limiting in any way.

### EXAMPLE 1

FIG. 2 demonstrates one particular example of suitable materials and techniques for creating an electrospun fiber having nanochannels 110. In this particular example, 10 wt % PLG 82:18 was dissolved in DCM and left to mix for at least 24 hours. The polymer solution was then electrospun into nanofiber sheets with a 35 cm needle tip-to-collector distance, a 13 kV positive lead, a −6 kV negative lead, a 5 mL/hr flow rate, and at 57% relative humidity. As described above, DCM is immiscible in water. Accordingly, electrospinning with this solvent at a high relative humidity causes a phase separation between the solvent and the polymer being electrospun, which creates the nanochannels 110 once the solvent is dissolved. As described above, as one example, the PLG can be electrospun into a shell structure (e.g., a tubular structure or a sheet) to house a conductive element and one or more agents. The electrospun PLG structure could also be loaded or combined with a conductive element and one or more agents, as described above. The electrospun PLG structure could also be processed into fragments and/or clusters, as described above.

### EXAMPLE 2

FIG. 3 demonstrates one particular example of suitable materials and techniques for fabricating an electrospun nanofiber structure with conductive particles. In this particular example, 100 wt % Ta nanoparticles of 10 wt % PLG 82:18 was dissolved in DCM and left to mix for at least 24 hours. The polymer solution was then electrospun into nanofiber sheets with a 25 cm needle tip-to-collector distance, a 12 kV positive lead, a −6 kV negative lead, a 2.5 mL/hr flow rate, and at 62% relative humidity. Accordingly, electrospinning the combination of the PLG and the Ta nanoparticles results in a nanofiber that is loaded with the Ta, which serves as the conductive element. The electrospun Ta-loaded PLG structure could also be combined with one or more agents as described above. The electrospun Ta-loaded PLG structure could also be processed into fragments and/or clusters as described above.

### EXAMPLE 3

FIG. 4 demonstrates one particular example of suitable materials and techniques for fabricating an electrospun nanofiber including a shell enclosing a conductive polymer structure. In this particular example, 1000 wt % Ta nanoparticles of 6 wt % PLG 82:18 was dissolved in hexaflouroisoproponol (HFIP) and left to mix for at least 24 hours. Additionally, 10 wt % PLG 82:18 was dissolved in DCM and left to mix for at least 24 hours. The polymer solutions were then electrospun into nanofiber sheets using concentric 20-gauge and 16-gauge needle tips. The solution containing Ta nanoparticles, PLG 82:18, and HFIP was spun using the inner, 20 gauge needle tip with a flow rate of 2 mL/hr. The 1000 wt % Ta nanoparticles serves to effectively provide a nearly solid metal core to the resulting electrospun nanofiber. The solution containing PLG 82:18 and DCM was spun using the outer, 16 gauge needle tip with a flow rate of 8 mL/hr. Further, the electrospinning was performed at 56% relative humidity using a 30 cm needle tip-to-collector distance, a 19.8 kV positive lead, and a −6.6 kV negative lead. Accordingly, the solution containing Ta, PLG 82:18, and HFIP spun from the inner needle forms the conductive polymer structure, and the solution containing PLG 82:18 and DCM forms the shell enclosing the conductive polymer structure. The electrospun nanofiber structure can then be further combined or loaded with one or more agents as described above.

### EXAMPLE 4

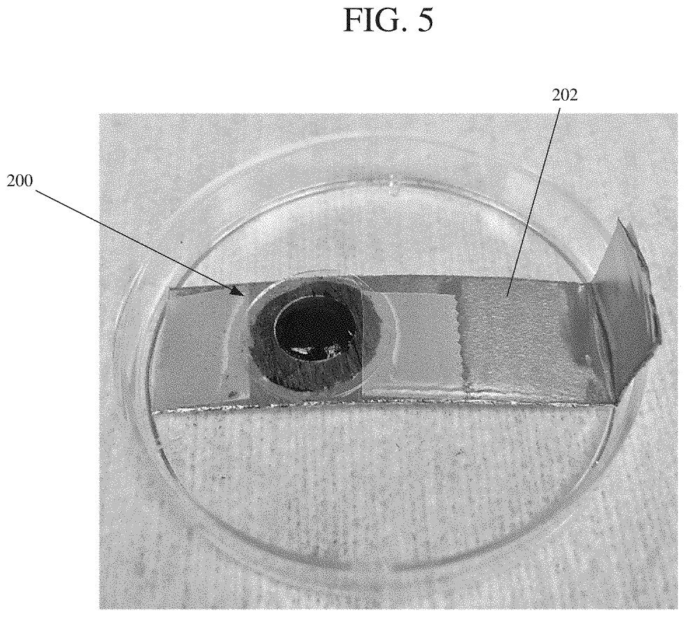

FIG. 5 demonstrates an example of an experimental setup for testing the electrospun nanofiber structures and compositions described herein. In this particular example, an electrospun nanofiber structure 200 including one or more agents (e.g., plasmids) that was fabricated using described techniques is shown. The electrospun nanofiber structure 200 is electrically coupled to a negative electrode 202. The electrospun nanofiber structure 200 can then be positioned topically against the subject and a corresponding positive counter-electrode can be applied to (e.g., inserted through the skin of) the subject to complete the circuit with the negative electrode 202. When the electrodes are coupled, the electric field is focused through the electrospun nanofiber structure 200 (e.g., via the nanochannels), inducing electroporation in the subject's cell membranes in the vicinity of the electrospun nanofiber structure 200. Accordingly, the one or more agents are delivered through the cell membranes' pores, resulting in the transfer of the one or more agents (e.g., plasmids) to the subject to, e.g., therapeutically treat the patient.

While the present disclosure has been illustrated by the description of exemplary embodiments thereof, and while the embodiments have been described in certain detail, it is not the intention of the Applicants to restrict or in any way limit the scope of the appended claims to such detail. Additional advantages and modifications will readily appear to those skilled in the art. Therefore, the disclosure in its broader aspects is not limited to any of the specific details, representative devices and methods, and/or illustrative examples shown and described. Accordingly, departures may be made from such details without departing from the spirit or scope of the Applicant's general inventive concept.

1. A conductive nanofiber comprising:
a shell electrospun from an insulating polymer, wherein the shell comprises a plurality of nanochannels therethrough;
a conductive element; and
an agent contained within the shell, wherein the agent comprises at least one of a therapeutic agent, a prophylactic agent, or a diagnostic agent;
wherein the conductive nanofiber is configured to deliver the agent when exposed to an electric field.

2. The conductive nanofiber of claim 1, wherein the conductive element comprises a conductive polymer fiber.

3. The conductive nanofiber of claim 2, wherein the conductive polymer fiber comprises polyaniline.

4. The conductive nanofiber of claim 1, wherein the agent comprises at least one of genetic material or genome editing machinery.

5. The conductive nanofiber of claim 4, wherein the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

6. The conductive nanofiber of claim 1, wherein the conductive nanofiber is in the form of a tubular structure.

7. The conductive nanofiber of claim 1, wherein the conductive nanofiber is in the form of a planar structure.

8. The conductive nanofiber of claim 7, wherein the planar structure is selected from the group consisting of a graft, a wound dressing, a muscle wrapping, and a hernia mesh.

9. The conductive nanofiber of claim 1, wherein the insulating polymer comprises polylactide-co-glycolide acid.

10. A method of treating a subject, the method comprising:
applying an electrospun nanofiber structure to the subject, wherein the electrospun nanofiber structure comprises a plurality of conductive nanofibers, each of the plurality of conductive nanofibers comprising:
a shell electrospun from an insulating polymer, wherein the shell comprises a plurality of nanochannels therethrough,
a conductive element, and
an agent contained within the shell, wherein the agent comprises at least one of a therapeutic agent, a prophylactic agent, or a diagnostic agent; and
applying an electric field to the electrospun nanofiber structure or the subject to cause the electrospun nanofiber structure to deliver the agent via electroporation of cell membranes of the subject.

11. The method of claim 10, wherein the conductive element comprises a conductive polymer fiber.

12. The method of claim 11, wherein the conductive polymer fiber comprises polyaniline.

13. The method of claim 10, wherein the agent comprises at least one of genetic material or genome editing machinery.

14. The method of claim 13, wherein the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

15. The method of claim 10, wherein the conductive nanofiber is in the form of a tubular structure.

16. The method of claim 10, wherein the conductive nanofiber is in the form of a planar structure.

17. The method of claim 16, wherein the planar structure is selected from the group consisting of a graft, a wound dressing, a muscle wrapping, and a hernia mesh.

18. The method of claim 10, wherein the insulating polymer comprises polylactide-co-glycolide acid.

19. A method of fabricating an electrospun nanofiber structure, the method comprising:
dissolving an insulating polymer in a first solvent, wherein the solvent is immiscible in water;
electrospinning the insulating polymer from an outer needle at a high relative humidity;
electrospinning a conductive polymer from an inner needle, wherein the inner needle is arranged coaxially with respect to the inner needle;
wherein the electrospun insulating polymer forms a shell of the electrospun nanofiber structure and the electrospun conductive polymer forms a core of the electrospun nanofiber structure, wherein the core is contained within the shell;
dissolving the solvent to form a plurality of nanochannels in the shell; and
applying an agent to the electrospun nanofiber structure, wherein the agent comprises at least one of a therapeutic agent, a prophylactic agent, or a diagnostic agent.

20. The method of claim 19, wherein the conductive polymer fiber comprises polyaniline.

21. The method of claim 19, wherein the agent comprises at least one of genetic material or genome editing machinery.

22. The method of claim 21, wherein the genetic material comprises at least one of DNA, RNA, cDNA, extrachromosomal DNA, messenger RNA, ribosomal RNA, transfer RNA, small interfering RNA, micro RNA, small nuclear RNA, small nucleolar RNA, piwi-interacting RNA, non-coding RNA, long noncoding RNA, or fragments or portions thereof.

23. The method of claim 19, wherein the conductive nanofiber is in the form of a tubular structure.

24. The method of claim 19, wherein the conductive nanofiber is in the form of a planar structure.

25. The method of claim 24, wherein the planar structure is selected from the group consisting of a graft, a wound dressing, a muscle wrapping, and a hernia mesh.
